(19) United States
(12) Patent Application Publication
(10) Pub. No.: US 2003/0055312 A1
(43) Pub. Date: Mar. 20, 2003

(54) Canine authentic intrauterine sound device

(76) Inventor: Genelle Coats, Pleasanton, CA (US)

Correspondence Address: Genelle Coats, 774 Neal Place, Pleasanton, CA, 94566, US

(21) Appl. No.: 09/953,217

(22) Filed: Sep. 17, 2001

(51) Int. Cl. A61M 21/00

(52) U.S. Cl. 600/28

(57) ABSTRACT

The Canine Authentic Intrauterine Sound Device (CAISD) provides a single, compact, easy to use device for dog owners. The CAISD will provide original, familiar, authentic canine intrauterine sounds to relieve the stress of separation for the new puppy and/or the stress of a canine that has experienced some kind of trauma. The CAISD, equipped with motion and sound sensitivity, will minimize sleep deprivation for both puppy and owner alike, thereby allowing the young new canine the opportunity to adapt to its new environment in as natural a manner as possible.

### CROSS-REFERENCE TO RELATED APPLICATIONS

Not applicable

### STATEMENT REGARDING FEDERALLY SPONSORED RESEARCH OR DEVELOPMENT

Not applicable

### REFERENCE TO A MICROFICHE APPENDIX

Not Applicable

### BACKGROUND—FIELD OF INVENTION

This invention relates to the use of canine authentic intrauterine sounds as a stress reducer for new puppies.

### BACKGROUND—DESCRIPTION OF PRIOR ART

Not applicable

### SUMMARY

This original and novel canine authentic intrauterine sound device would replace the most widely suggested possible resolution to a howling/crying new puppy, the ticking clock and hot water bottle.

### DESCRIPTION OF SEVERAL VIEWS OF THE DRAWINGS

The drawings closely related figures have been numbered as follows:

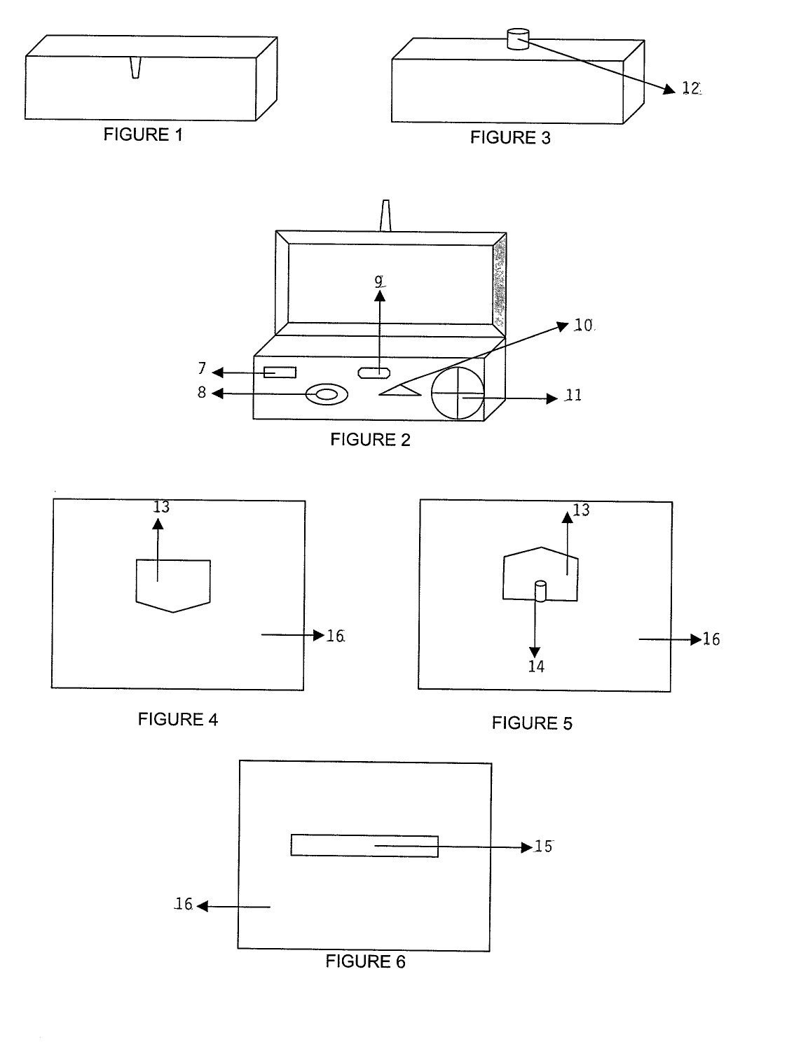

1. FIG. 1 shows plastic box with removable lid with coin twist slot opening.

2. FIG. 2 shows inner workings of the plastic box with computer chip, speaker, motion and sound sensors, and battery chamber.

3. FIG. 3 shows the on/off/volume knob at the bottom of the plastic box.

4. FIG. 4 shows front view of stuffed pillow with small velcro flap closure. (closed)

5. FIG. 5 shows front view of stuffed pillow with small velcro flap closure (opened) to reveal button hole and on/off/volume knob.

6. FIG. 6 shows back of stuffed pillow with linear velcro closure providing access to the plastic box in order to add/remove batteries.

### REFERENCE NUMERALS IN DRAWINGS

7 computer chip

8 speaker

9 motion sensor

10 sound sensor

11 battery chamber

12 on/off/volume knob

13 small front velcro closure

14 button hole opening for on/off volume knob

15 back linear velcro closure to box and batteries

### DETAILED DESCRIPTION OF INVENTION

The idea for a Canine Authentic Intrauterine Sound Device came to me while at my niece's baby shower. I overheard the young women in attendance discussing the use of sounds from the mother's womb that would calm a baby during sleeptime. I thought surely this could work for puppies.

I called Autumn P. Davidson, DVM, DACVIM, Director, Veterinary Clinic for Guide Dogs For The Blind and reproductive specialist for the University of California, Davis, Veterinary School. Additionally, I called upon the expertise of John Rezendes, (need John's business card) computer software and sound recording specialist, to assist in recording the canine intrauterine sounds I needed.

On Apr. 10, 2001, at the Guide Dogs for the Blind veterinary clinic, Dr. Davidson presented Jetta, a 4-year old Black Labrador Retriever who was due to deliver her litter of new puppies on Apr. 17, 2001. We were successful in recording, for the first time, authentic canine intrauterine sounds. (Disk of the recorded sounds is enclosed)

This original, novel sound will be digitized and looped to run approximately 5 minutes at a time and will be applied to a computer chip.

The computer chip (7), along with a small speaker (8) and both motion (9) and sound (10) sensors will be placed in a small plastic box with a removable lid. (FIG. 2)

The device will also need 4 AA batteries (11) to operate. (FIG. 2)

The on/off/volume knob (12) will be located at the bottom exterior of the plastic box. (FIG. 3)

The plastic box (FIG. 1) will then be placed in an approximate 10″×7″ stuffed pillow. (FIG. 4)

The front of the pillow will have a small velcro opening/closure (13). (FIG. 4)

You lift the closure to have access to the on/off/volume knob (12). (FIG. 5)

The back of the pillow will have a linear velcro opening/closure (15) that allows the operator access to the plastic box in order to add/remove the batteries. (FIG. 6)

In order to present the sound as intended, the Canine Authentic Intrauterine Sound Device should be placed either in the kennel or sleep area of the puppy.

The operator turns on the CAISD and chooses the volume of choice.

The CAISD will run for approximately 5 minutes or continue to run as long as there is movement or sounds being made by the puppy.

Once the puppy is quiet and calm the CAISD will automatically shut off

If the puppy becomes restless and moves, the motion sensor will activate the canine intrauterine sound until the puppy stops.

The sound sensor will also activate the CAISD in the same manner, if the puppy were to begin to howl or cry.

The Canine Authentic Intrauterine Sound Device, is a novel, new way of reducing the stress that puppies exhibit upon the separation from their mother and littermates and would replace the ticking clock and hot water bottle alternative suggested by vets and pet stores.

An Additional advantage of the Canine Authentic Intrauterine Sound Device would be to aid in minimizing/reducing the stress and fear often associated with canines during trauma. This familiar, soothing sound will help to calm an otherwise agitated or frightened canine and keep them from doing further harm to themselves or others.

I claim that my invention:

1. the Canine Authentic Intrauterine Sound Device (CAISD) uses authentic, original intrauterine sounds of a pregnant female dog recorded specifically for my invention.
